(19) United States
(12) Patent Application Publication
(10) Pub. No.: US 2024/0345026 A1
(43) Pub. Date: Oct. 17, 2024

(54) MAGNETIC FILM FUEL DETECTOR

(71) Applicants: Saudi Arabian Oil Company, Dhahran (SA); King Abdullah University of Science and Technology, Thuwal (SA)

(72) Inventors: Maha Nour, Thuwal (SA); Abdullah Hassan Bukhamsin, Thuwal (SA); Esraa Fakeih, Thuwal (SA); Sumana Bhattacharjee, Thuwal (SA); Khaled Nabil Salama, Thuwal (SA)

(21) Appl. No.: 18/135,533

(22) Filed: Apr. 17, 2023

(51) Int. Cl. G01N 27/74; G01N 33/44

(52) U.S. Cl. CPC G01N 27/74; G01N 33/442; G01N 33/0003

(57) ABSTRACT

A system and a method for detecting aromatic compounds mixed with aliphatic compounds are provided. As exemplary system includes a lower substrate including a magnet and a magnetic film comprising a magnetic particle filler in a polymer matrix disposed over the lower substrate. An upper substrate is disposed over the magnetic film wherein the upper substrate includes an opening extending to the magnetic film.

### TECHNICAL FIELD

This disclosure relates to a system and method for detecting hydrocarbon fuels blended in paraffin-based lubricants.

### BACKGROUND

Fuel smuggling is a growing problem in international commerce. To avoid detection, smugglers may blend hydrocarbon fuels with lubrication oils and greases that are based on paraffinic compounds. Generally, hydrocarbon fuels contain low molecular weight aromatic compounds that can be used to detect the illicit compounds.

### SUMMARY

An embodiment described herein provides a system for detecting aromatic compounds mixed with aliphatic compounds. The system includes a lower substrate including a magnet and a magnetic film comprising a magnetic particle filler in a polymer matrix disposed over the lower substrate. An upper substrate is disposed over the magnetic film wherein the upper substrate includes an opening extending to the magnetic film.

Another embodiment described herein provides a method for detecting aromatic compounds mixed with aliphatic compounds. The method includes placing a sample of a test fluid on a magnetic film through an opening in an upper substrate, wherein the magnetic film is under stress from a magnet in a lower substrate below the magnetic film. A timer is started when the sample contacts the magnetic film. The timer is stopped when the magnetic film breaks. A difference in time between starting the timer and stopping the timer provides a test time. The test time is used to determine if an aromatic compound is present in the test fluid.

### BRIEF DESCRIPTION OF DRAWINGS

FIG. 1A is a perspective view of an aromatics detector based on a magnetic film.

FIG. 1B is a top view of the aromatics detector.

FIG. 1C is a side, cross-sectional view of the aromatics detector.

FIG. 2 is a drawing of a magnetic film having a polymer matrix that includes magnetic particles.

FIG. 3 is a method of making a magnetic film from a cyclic olefin copolymer (COC).

FIG. 4 is a method of using an aromatics detector to determine the presence of aromatics mixed into a matrix of aliphatic compounds.

### DETAILED DESCRIPTION

Embodiments described herein provide a system and method for determining the presence of aromatic compounds in lubricants and greases based on paraffinic (aliphatic) compounds. The detection system, herein termed an aromatic detector, is based on the differential solubility of a polymer between aromatic compounds and aliphatic compounds.

A magnetic powder is blended with a polymer to form a magnetic film. The magnetic film is placed over a magnet in a test apparatus, which places the magnetic film under stress. To test for aromatic compounds in a test fluid, a sample of the test fluid is placed on the magnetic film and, if aromatic compounds are present, the polymer will start to disintegrate. The stress force on the magnetic polymer film from the magnet will then break the film, indicating the presence of aromatic compounds in the test fluid.

For some polymers, contact with a material that is a poor solvent may result in stress cracking, or slower dissolution, leading to breaking of the film under stress. However, the dissolution or disintegration rate will be faster for solvents. In these embodiments, the time to break the film after the application of the test fluid can be compared to the time to break the film after the application of a control fluid. For example, a paraffinic fluid or paraffinic oil that does not contain aromatics may be used as the control fluid.

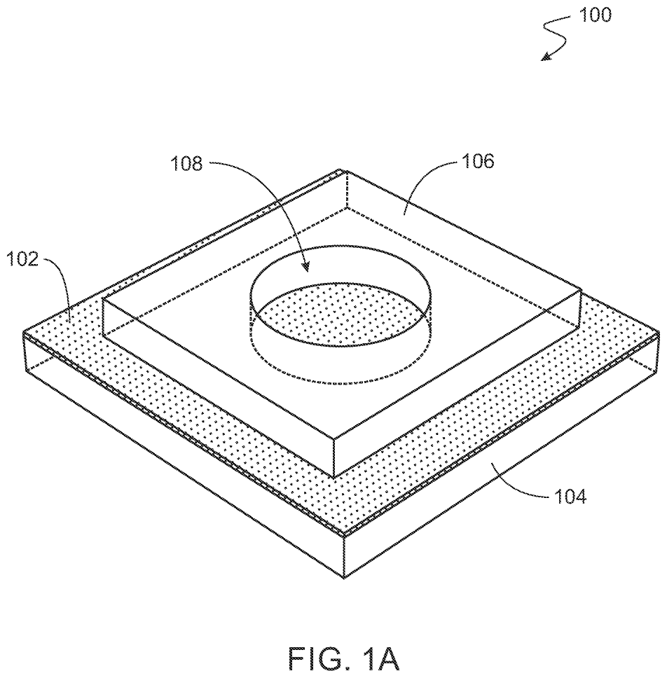

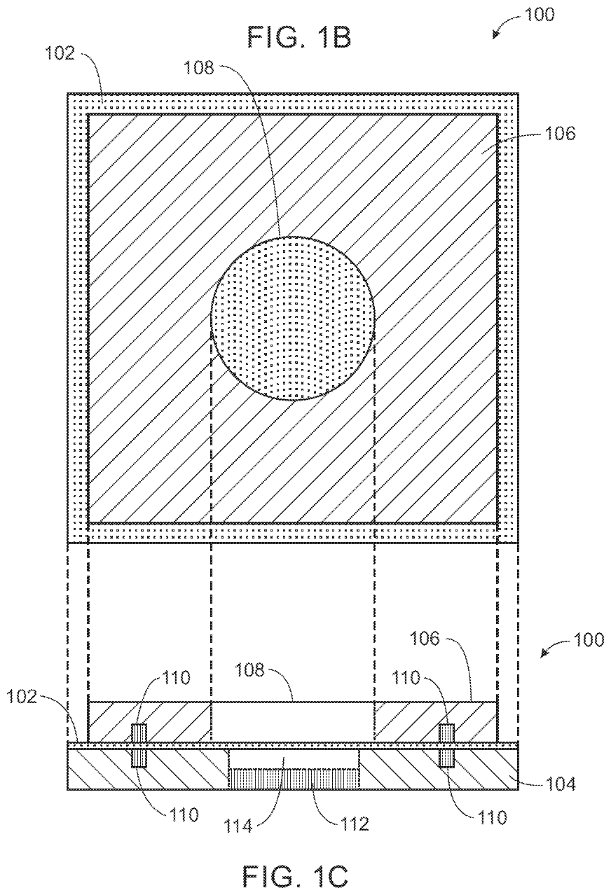

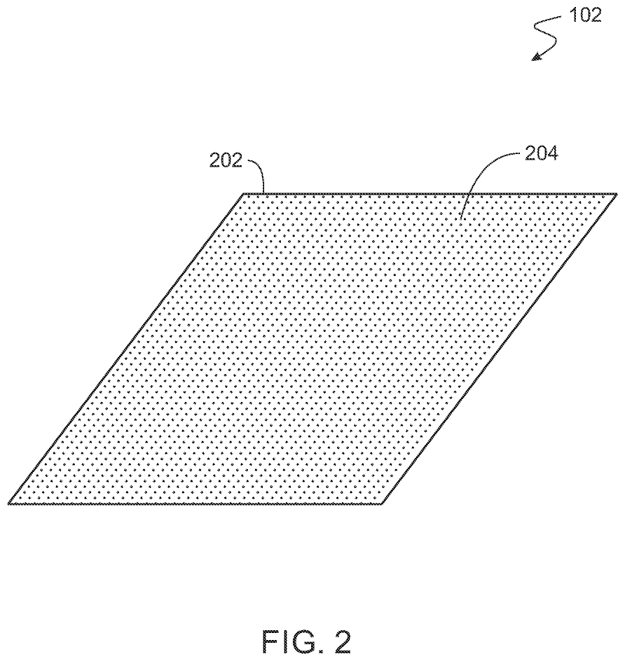

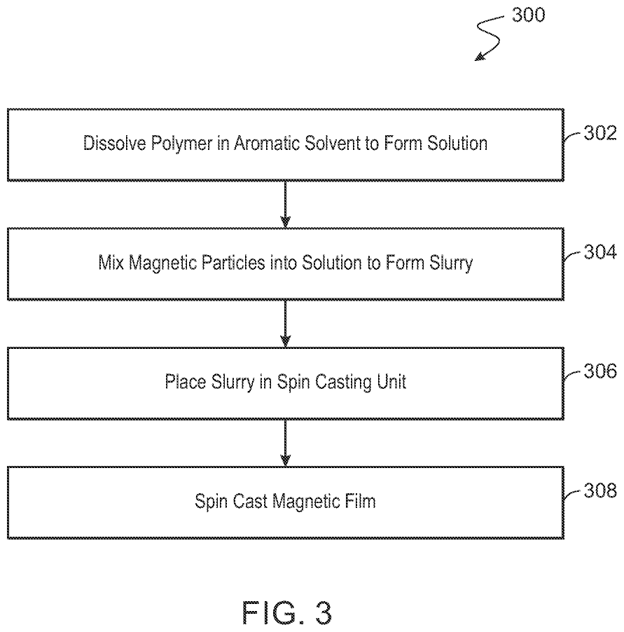

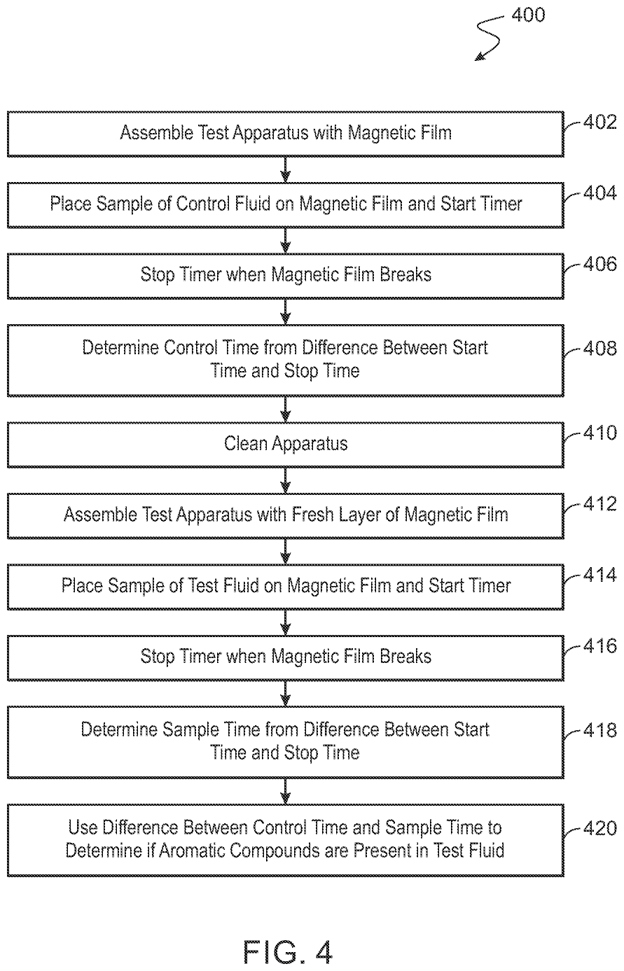

The device, or aromatics detector, is discussed with respect to FIGS. 1A-1C. The magnetic film is discussed with respect to FIG. 2. A method for making the magnetic film is discussed with respect to FIG. 3. The test procedure for using the aromatics detector is discussed with respect to FIG. 4.

FIG. 1A is a perspective view of an aromatics detector 100 based on a magnetic film 102. In the aromatics detector 100, the magnetic film 102 is placed between a lower substrate 104 and an upper substrate 106. An opening 108 in the upper substrate 106 exposes the magnetic film 102, allowing a sample of a fluid to be placed on the magnetic field for testing. The sample may be about 0.3 mL, about 0.5 mL, about 1 mL, or about 2 mL, or more. The amount of sample used may depend on the likely concentration of aromatics in the solution.

In various embodiments, the lower substrate 104 comprises polymethyl methacrylate, acetal copolymer, acetal homopolymer, nylon, polytetrafluoroethylene (PTFE), polyvinylidene fluoride copolymer, or glass, or any combinations thereof. In various embodiments, the upper substrate 106 comprises polymethyl methacrylate, acetal copolymer, acetal homopolymer, nylon, polytetrafluoroethylene (PTFE), polyvinylidene fluoride copolymer, or glass, or any combinations thereof. The lower substrate 104 and the upper substrate 106 may be made from different materials.

FIG. 1B is a top view of the aromatics detector 100. The top view shows the opening 108 through which the magnetic film 102 is accessible. During a test, a sample of the test fluid is placed on the magnetic film 102 through the opening 108 in the upper substrate 106.

FIG. 1C is a side, cross-sectional view of the aromatics detector 100. As can be seen in this view, the magnetic film 102 is held in place between the lower substrate 104 and the upper substrate 106. In some embodiments, magnets 110 built into the lower substrate 104 and the upper substrate 106 are used to assist with alignment during assembly. In various embodiments, the lower substrate 104 and the upper substrate 106 are held together with mechanical clips, adhesives, or solvent bonding.

In some embodiments, a magnet 112 is mounted in the lower substrate 104, for example, in an opening 114 located below the magnetic film 102. The opening 114 ensures that the magnetic film 102 is unsupported under the sample, creating stress on the magnetic film 102 from the magnet 112. The diameter of the magnet 112 and the proximity to the magnetic film 102 can be selected, for example, based on the polymer and magnetic particles used to form the magnetic film 102. In various embodiments, the magnet 112 includes NdFeB, CoSm, and the like. Similar materials may be used for the magnets 110 used for alignment. The openings 108 and 114 can be fabricated using a CO₂ laser or a mechanical process.

As materials, such as aromatic compounds, attack the magnetic film, it will fail under the stress from the magnet 112. As discussed below, the time to failure can be used to indicate the presence of the aromatic compounds from fuels.

FIG. 2 is a drawing of a magnetic film 102 having a polymer matrix 202 that includes magnetic particles 204. In various embodiments, the polymer matrix 202 is a cyclic olefin copolymer (COC), a poly(acrylonitrile butadiene styrene) (ABS), a polyphenylene oxide (PPO), and the like. The choice of polymer 202 may be used to control the sensitivity of the test, depending on the susceptibility of the polymer matrix 202 to dissolution or stress cracking from components of the sample.

In various embodiments, the magnetic particles 204 include neodymium-iron-boron (NdFeB), magnetite ((Fe²⁺Fe³⁺)₂O₄), iron (Fe), cobalt, nickel, and the like. The choice of the magnetic particles 204 may also be used to control the sensitivity, as particles made from stronger magnetic materials may increase the stress on the magnetic film, for example, NdFeB may be stronger than iron, which may be stronger than magnetite. Further, the size of the magnetic particles 204 may be selected based on the thickness of the magnetic film 102 expected. In various embodiments, the magnetic particles 204 may be less than about 200 nm in size, less than about 500 nm in size, less than about 1000 nm in size, or less than about 5000 nm in size. Larger particles may be used in thicker films, for example, made from more susceptible polymers

FIG. 3 is a method 300 of making a magnetic film from a cyclic olefin copolymer (COC). At block 302, the COC is dissolved in toluene to form a solution. At block 304, the solution is mixed with the magnetic powder, such as NdFeB, to form a slurry. At block 306, the slurry is placed in the spin-casting unit, for example, being injected through a syringe onto a spinning substrate. At block 308, the film is spin cast by being flowed onto a rotating platform. The rotating speed and the concentration of the COC in the solvent controls the thickness of the COC layer. For example, the concentration of the COC in the toluene solvent may range from about 10% to about 30%, or about 20%. The rotational speed of the spin-casting unit can be about 500 to about 5000 rpm, or about 1000 rpm to about 4000 rpm, or about 2000 rpm. The solvent is then evaporated through a baking process that decreases the evaporation time, forming a solid film from the solution. The magnetic film is then magnetized, for example, in an electromagnetic field. The orientation of the magnetic field may be selected based on the magnetic field of the magnet 112 (FIG. 1) used for the test.

In some embodiments, the magnetic film is formed by plastics processing techniques. For example, the magnetic powder may be compounded with a plastic resin in a compounding extruder to form pellets. The pellets are then used to form a film, for example, in a sheet extruder or a blown film extruder. The film is then cut into the final size and shape, for example, as shown with respect to FIGS. 1A-1C, and FIG. 2, and then magnetized to form the final magnetic film for testing.

FIG. 4 is a method 400 of using an aromatics detector to determine the presence of aromatics mixed into a matrix of aliphatic compounds. The method begins at block 402 with assembling a test apparatus that includes a magnetic film, as described herein. The test apparatus is assembled by placing the magnetic film between two substrates. A lower substrate, placed under the magnetic film, includes a magnet to place the magnetic film under stress. An upper substrate, placed over the magnetic film, has an opening leading to the magnetic film.

At block 404, a sample of a control fluid is placed on the magnetic film and a timer is started. At block 406, the timer is stopped when the magnetic film breaks. At block 408, the time for the control fluid to break the magnetic film, or the control time, is calculated by taking the time difference between the starting time and the stopping time of the timer.

At block 410, the apparatus is cleaned. For example, the upper substrate is separated from the lower substrate and the broken magnetic film is removed. Any excess control fluid is wiped away or removed by cleaning. In some embodiments, the test apparatus is a one-time use apparatus, and is discarded after the test is complete. In these embodiments, a single determination of the control time may be made and supplied with the test apparatus.

At block 412, the test apparatus is reassembled with a fresh layer of magnetic film. At block 414, a sample of test fluid is placed on the magnetic film and the timer is started. At block 416, the timer is stopped when the magnetic film breaks. At block 418, the time for the test fluid to break the magnetic film, or the test time, is calculated by taking the time difference between the starting time and the stopping time of the timer.

At block 420, a difference between the control time and the sample time is used to determine if aromatic compounds are present in the test fluid, indicating the contamination of the paraffinic compounds. For example, the sample time will be lower than the control time, as the polymer is more susceptible to dissolution or stress cracking from aromatic compounds than from aliphatic compounds.

The test procedure is not limited to the specific steps shown in the blocks above. For example, if the polymer used is not susceptible to dissolution by aliphatic compounds, the test may be implemented with no control fluid. In this example, a few milliliters of the test sample is placed on the magnetic film through the opening of the upper substrate, and the leakage of the liquid through the magnetic film is observed visually.

### Examples

The formation of a COC film was tested. The COC was purchased from TOPAS advanced polymers of Florence, KY, USA. The great selected was 50131-10 which had 3 mm nominal granule size.

To form the film, the COC was dissolved in toluene at a 20% volume volume ratio, following the initial solution. The solution was dropped onto the spinning turntable of the spin-casting unit, while the turntable was operated at 2000 RPM for 60 seconds. This formed a film of about 1.5 μm in thickness.

### Embodiments

An embodiment described herein provides a system for detecting aromatic compounds mixed with aliphatic compounds. The system includes a lower substrate including a magnet and a magnetic film comprising a magnetic particle filler in a polymer matrix disposed over the lower substrate. An upper substrate is disposed over the magnetic film wherein the upper substrate includes an opening extending to the magnetic film.

In an aspect, the system includes an opening over the magnet in the center of the lower substrate, wherein the opening is between the magnet and the magnetic film.

In an aspect, the lower substrate includes glass. In an aspect, the lower substrate includes acetal copolymer, acetal homopolymer, nylon, polytetrafluoroethylene (PTFE), or polyvinylidene fluoride, or any combination thereof.

In an aspect, the polymer matrix includes a cyclic olefin copolymer (COC). In an aspect, the polymer matrix includes poly(acrylonitrile butadiene styrene) (ABS). In an aspect, the polymer matrix includes polyphenylene oxide (PPO).

In an aspect, the magnetic particles include neodymium-iron-boron (NdFeB). In an aspect, the magnetic particles include magnetite ((Fe²⁺Fe³⁺)₂O₄). In an aspect, the magnetic particles include iron (Fe).

In an aspect, the upper substrate includes glass. In an aspect, the upper substrate includes acetal copolymer, acetal homopolymer, nylon, polytetrafluoroethylene (PTFE), or polyvinylidene fluoride, or any combination thereof.

Another embodiment described herein provides a method for detecting aromatic compounds mixed with aliphatic compounds. The method includes placing a sample of a test fluid on a magnetic film through an opening in an upper substrate, wherein the magnetic film is under stress from a magnet in a lower substrate below the magnetic film. A timer is started when the sample contacts the magnetic film. The timer is stopped when the magnetic film breaks. A difference in time between starting the timer and stopping the timer provides a test time. The test time is used to determine if an aromatic compound is present in the test fluid.

In an aspect, the method includes assembling a test apparatus with a layer of magnetic film between two substrates, wherein an upper substrate has an opening to the magnetic film, and the lower substrate has a magnet below the magnetic film.

In an aspect, the method includes removing the magnetic film from the test apparatus once it breaks. The test apparatus is cleaned and reassembled with layer of magnetic film between the upper substrate and the lower substrate. In an aspect, the method includes placing a sample of control fluid on the magnetic film through the opening in the upper substrate. The timer is started when the sample contacts the magnetic film and stopped when the magnetic film breaks. A measured time is determined as the difference in time between starting and stopping the timer. The measured time is used as a control time.

In an aspect, the method includes comparing the test time to the control time to determine if an aromatic compound is present in the test fluid.

In an aspect, the method includes forming the magnetic film by dissolving a polymer in an aromatics solvent to form a solution. Magnetic particles are mixed into the solution to form a slurry and the slurry is placed in a spin casting unit to cast the magnetic film.

In an aspect, the polymer includes a cyclic olefin copolymer.

In an aspect, the magnetic particles include neodymium-iron-boron (NdFeB).

In an aspect, the magnetic film is formed by compounding a filler including magnetic particles into a polymer, and extruding the polymer into a film. In an aspect, the polymer includes an acrylonitrile butadiene styrene (ABS) copolymer. In an aspect, the magnetic particles include iron.

Other implementations are also within the scope of the following claims.

What is claimed is:

1. A system for detecting aromatic compounds mixed with aliphatic compounds, comprising:
a lower substrate comprising a magnet;
a magnetic film comprising a magnetic particle filler in a polymer matrix disposed over the lower substrate; and
an upper substrate disposed over the magnetic film, wherein the upper substrate comprises an opening extending to the magnetic film.

2. The system of claim 1, comprising an opening over the magnet in the center of the lower substrate, wherein the opening is between the magnet and the magnetic film.

3. The system of claim 1, wherein the lower substrate comprises glass.

4. The system of claim 1, wherein the lower substrate comprises acetal copolymer, acetal homopolymer, nylon, polytetrafluoroethylene (PTFE), or polyvinylidene fluoride, or any combination thereof.

5. The system of claim 1, wherein the polymer matrix comprises a cyclic olefin copolymer (COC).

6. The system of claim 1, wherein the polymer matrix comprises poly(acrylonitrile butadiene styrene) (ABS).

7. The system of claim 1, wherein the polymer matrix comprises polyphenylene oxide (PPO).

8. The system of claim 1, wherein the magnetic particles comprise neodymium-iron-boron (NdFeB).

9. The system of claim 1, wherein the magnetic particles comprise magnetite ((Fe²⁺Fe³⁺)₂O₄).

10. The system of claim 1, wherein the magnetic particles comprise iron (Fe).

11. The system of claim 1, wherein the upper substrate comprises glass.

12. The system of claim 1, wherein the upper substrate comprises acetal copolymer, acetal homopolymer, nylon, polytetrafluoroethylene (PTFE), or polyvinylidene fluoride, or any combination thereof.

13. A method for detecting aromatic compounds mixed with aliphatic compounds, comprising:
placing a sample of a test fluid on a magnetic film through an opening in an upper substrate, wherein the magnetic film is under stress from a magnet in a lower substrate below the magnetic film;
starting a timer when the sample contacts the magnetic film;
stopping the timer when the magnetic film breaks, wherein a difference in time between starting the timer and stopping the timer provides a test time; and
using the test time to determine if an aromatic compound is present in the test fluid.

14. The method of claim 13, comprising assembling a test apparatus with a layer of magnetic film between two substrates, wherein an upper substrate has an opening to the magnetic film, and the lower substrate has a magnet below the magnetic film.

15. The method of claim 14, comprising:
removing the magnetic film from the test apparatus once it breaks;
cleaning the test apparatus; and
reassembling the test apparatus with a layer of magnetic film between the upper substrate and the lower substrate.

16. The method of claim 15, comprising:
placing a sample of control fluid on the magnetic film through the opening in the upper substrate;
starting the timer when the sample contacts the magnetic film;
stopping the timer when the magnetic film breaks to obtain a measured time as the difference in time between starting and stopping the timer; and
using the measured time as a control time.

17. The method of claim 16, comprising comparing the test time to the control time to determine if an aromatic compound is present in the test fluid.

18. The method of claim 13, comprising forming the magnetic film by:
dissolving a polymer in an aromatics solvent to form a solution;
mixing magnetic particles into the solution to form a slurry;
placing the slurry in a spin casting unit; and
casting the magnetic film.

19. The method of claim 18, wherein the polymer comprises a cyclic olefin copolymer.

20. The method of claim 18, wherein the magnetic particles comprise neodymium-iron-boron (NdFeB).

21. The method of claim 13, comprising forming the magnetic film by:
compounding a filler comprising magnetic particles into a polymer; and
extruding the polymer into a film.

22. The method of claim 21, wherein the polymer comprises an acrylonitrile butadiene styrene (ABS) copolymer.

23. The method of claim 21, wherein the magnetic particles comprise iron.
